(19) United States
(12) Patent Application Publication
(10) Pub. No.: US 2016/0199823 A1
(43) Pub. Date: Jul. 14, 2016

(54) SELECTIVE ZEOLITE SUPPORTED CATALYSTS FOR PROPANE AND BUTANE DEHYDROGENATION

(71) Applicant: SAUDI BASIC INDUSTRIES CORPORATION, Riyadh (SA)

(72) Inventor: Zeeshan Nawaz, Riyadh (SA)

(73) Assignee: SAUDI BASIC INDUSTRIES CORPORATION, Riyadh (SA)

(21) Appl. No.: 14/913,073

(22) PCT Filed: Aug. 20, 2014
(86) PCT No.: PCT/IB2014/063993

§ 371 Date: Feb. 19, 2016

Related U.S. Application Data
(60) Provisional application No. 61/868,340, filed on Aug. 21, 2013.

(51) Int. Cl. B01J 29/85; C07C 5/333; B01J 37/02; B01J 37/16; B01J 37/08

(52) U.S. Cl. CPC B01J 29/85; B01J 37/16; B01J 37/08; B01J 37/0201; B01J 37/0236; C07C 5/3337; B01J 2229/20

(57) ABSTRACT

Selective supported catalysts compositions for alkane dehydrogenation are described, the catalyst compositions containing platinum, a tin component, a potassium component, one or more additional promoter elements, and a support. Also described are methods for the preparation of such catalyst compositions and methods for the use of such catalyst compositions in the conversion of alkanes to olefins.

### BACKGROUND

1. Technical Field

The present disclosure relates to a catalyst, and specifically to multi-promoter, platinum based silicoaluminophosphate (SAPO) molecular sieve zeolite supported catalysts suitable for propane and/or butane dehydrogenation to olefins and/or corresponding olefins.

2. Technical Background

Catalytic dehydrogenation of alkanes is a common step in the production of olefins, such as, for example, propylene. Oxidative dehydrogenation processes are exothermic and can suffer from low selectivity and inferior olefins quality. Direct dehydrogenation processes can suffer from equilibrium limitations and the need for high temperatures to obtain desirable alkene yields. As such high temperatures can result in thermally cracking, therefore, the catalyst chemistry is important in controlling dehydrogenation reactions. The selection of an appropriate catalyst can significantly affect the conversion, selectivity, and olefin yield rate in dehydrogenation reactions. Most conventional catalyst materials are also subject to deterioration with use and/or age.

While much effort has been applied to the development and use of dehydrogenation catalysts, a need still exists for a catalyst materials that can provide one or more or improved stability, conversion, selectivity, and olefin yield rate. Thus, there is a need to address these and other shortcomings associated with conventional dehydrogenation catalyst materials. These needs and other needs are satisfied by the compositions and methods of the present disclosure.

### SUMMARY

In accordance with the purpose(s) of the invention, as embodied and broadly described herein, this disclosure, in one aspect, relates to a silicoaluminophosphate (SAPO) molecular sieve zeolite supported catalysts, platinum based catalysts, and specifically to a multi-promoted components suitable for propane and/or butane selective dehydrogenation.

In one aspect, the present disclosure provides a dehydrogenation catalysts composition from about 0.1 wt. % to about 1 wt. % platinum, based on the total amount of the catalyst compositions; a base promoter comprising a tin component and a potassium component; at least one additional promoter comprising a lanthanum component, a zinc component, a calcium component, a magnesium component, or a combination thereof.

In one aspect, a silicoaluminophosphate (SAPO) molecular sieves zeolites are used as supports for dehydrogenation catalysts.

### BRIEF DESCRIPTION OF THE FIGURES

The accompanying FIGURES, which are incorporated in and constitute a part of this specification, illustrate several non-limiting aspects and together with the description serve to explain the principles of the invention.

FIG. 1 illustrates the X-ray diffraction pattern of a Pt—Sn—K/Modified-SAPO-34 (Catalyst J) and a Scanning Electron Micrograph image of a Modified-SAPO-34 support.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise. Thus, for example, reference to “a ketone” includes mixtures of two or more ketones.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. “About” and “substantially” are intended to include the degree of error associated with measurement of the particular quantity based upon the equipment available at the time of filing the application.

As used herein, the terms “optional” or “optionally” means that the subsequently described event or circumstance can or can not occur, and that the description includes instances where said event or circumstance occurs and instances where it does not. For example, the phrase “optionally substituted alkyl” means that the alkyl group can or can not be substituted and that the description includes both substituted and unsubstituted alkyl groups.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

References in the specification and concluding claims to parts by weight of a particular element or component in a composition or article denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “alkoxy” as used herein is an alkyl group bound through a single, terminal ether linkage; that is, an “alkoxy” group may be defined as —OR where R is alkyl as defined above. A “lower alkoxy” group is an alkoxy group containing from one to six carbon atoms.

The term “alkenyl group” as used herein is a hydrocarbon group of from 2 to 24 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” as used herein is an aryl group having an alkyl, alkynyl, or alkenyl group as defined above attached to the aromatic group. An example of an aralkyl group is a benzyl group.

The term “hydroxyalkyl group” as used herein is an alkyl, alkenyl, alkynyl, aryl, aralkyl, cycloalkyl, halogenated alkyl, or heterocycloalkyl group described above that has at least one hydrogen atom substituted with a hydroxyl group.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

The term “carboxylic acid” as used herein is represented by the formula —C(O)OH.

The term “aldehyde” as used herein is represented by the formula —C(O)H.

The term “keto group” as used herein is represented by the formula —C(O)R, where R is an alkyl, alkenyl, alkynyl, aryl, aralkyl, cycloalkyl, halogenated alkyl, or heterocycloalkyl group described above.

The term “carbonyl group” as used herein is represented by the formula C═O.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfo-oxo group” as used herein is represented by the formulas —S(O)₂R, —OS(O)₂R, or, —OS(O)₂O R, where R can be hydrogen, an alkyl, alkenyl, alkynyl, aryl, aralkyl, cycloalkyl, halogenated alkyl, or heterocycloalkyl group described above.

As used herein, the term “promoter catalyst system” is intended to refer to a catalyst system comprising a promoter, unless specifically stated to the contrary. A promoter catalyst system can also be referred to as a promoted catalyst system, indicating the presence of a promoter in the catalyst system.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present disclosure provides dehydrogenation catalysts and compositions comprising such catalysts that can be useful in, for example, the conversion of alkanes to olefins and/or corresponding olefins. In one aspect, the catalysts composition can comprise a platinum based dehydrogenation catalyst. In another aspect, the catalyst composition can comprise tin, and potassium, together with one or more promoters comprising lanthanum, calcium, zinc, and/or magnesium. In yet another aspect, the catalyst composition comprises a zeolite support.

In various aspects, the catalyst composition comprises a supported catalyst. In one aspect, the support comprises a zeolite, such as, for example, a silicoaluminophosphate (SAPO) molecular sieve support. In another aspect, the support comprises a molecular sieve support, such as, for example, AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, ALPO-17, ALPO-18, ALPO-34, SAPO-5, SAPO-17, SAPO-18, SAPO-34, SAPO-35, SBA-15, MCM-42, ZK-4, ZSM-2, ZSM-5, ZK-14, SAPO-42, ZK-21, ZK-22, ZK-5, ZK-20, zeolite A, hydroxysodalite, erionite, chabazite, zeolite T, gmelinite, clinoptilolite, alumina bind/surface coated zeolites, and/or substituted groups thereof. In a specific aspect, the catalyst composition comprises a SAPO-34 silicoaluminophosphate molecular sieve support. In still another aspect, the support can comprise a micro pore support. In another aspect, the support can comprise a slit-SAPO-34 support. In another aspect, the physical and chemical properties of the support can impact the stability and activity of the catalyst. In various aspects, such a support can exhibit one or more of the following: a specific surface area of at least about 250 m²/g; a pore volume of at least about 0.1 cm³/g; and/or an average particle size of about ˜2 p.m. In other aspects, any one or more properties of the support material can vary, for example, based on the method of preparation and one of skill in the art, in possession of this disclosure, could readily select an appropriate support material.

In one aspect, a SAPO-34 support can be prepared by contacting Al₂O₃, P₂O₅, SiO₂, TEA, and H₂O in a molar ratio of about 1:1:0.5:2:100. In one aspect, the silicoaluminophosphate (SAPO) support can be un-modified and used in, for example, a commercially available form. In another aspect, the silicoaluminophosphate (SAPO) support can be modified with an aluminum source compound, and/or alumina as a binder and/or surface coating. In one aspect, the support can be modified with a non-mesopore template. In yet another aspect, the support can be modified with a combined source of aluminum and silica. In still another aspect, the support can be modified with kaolin. In such an aspect, kaolin, a phosphorus source, and water can be mixed to form a uniform or substantially uniform crystallization solution. In one aspect, kaolin, P₂O₅, and de-ionized water can be contacted in a molar ratio of about 1.5:1:500, after which the mixture can be stirred, aged, autoclaved, filtered, washed, dried, and calcined at about 600° C. to form a silicoaluminophosphate molecular sieve zeolite material. Thus, in various aspects, the support can comprise conventional, unmodified SAPO-34, a modified SAPO-34, a SAPO-34 bound with alumina, or a combination thereof.

In one aspect, the silicoaluminophosphate material can exhibit a specific shape selective morphology suitable for use in the dehydrogenation of alkanes. In one aspect, the silicoaluminophosphate can comprise a slit morphology, for example, that enhances access opportunities for alkanes to reach/access active sites inside the pores.

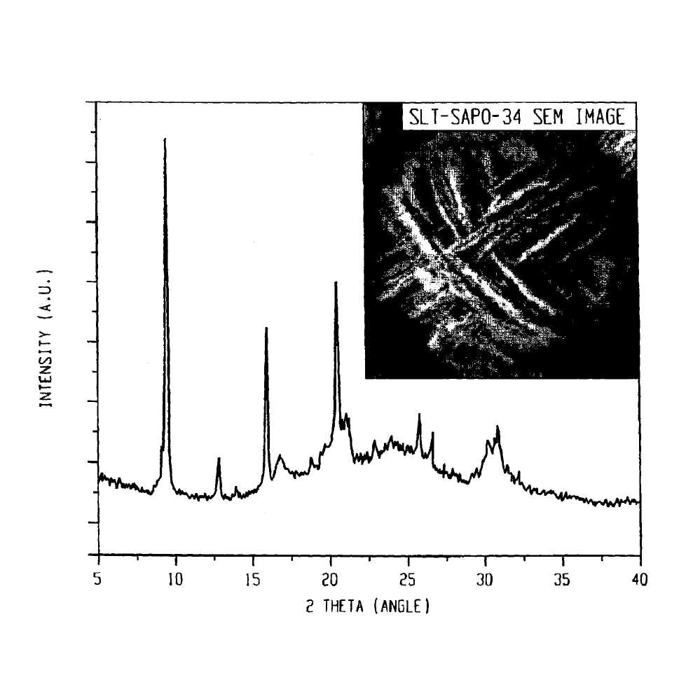

In one aspect, a catalyst can comprise Pt—Sn—K on a modified SAPO-34 support, as illustrated in FIG. 1. In such an aspect, the modified SAPO-34 support can comprise a slit morphology. In one aspect, all or substantially all of the support can be modified as described herein. In another aspect, only the surface of the support or a portion thereof can be modified.

The catalyst composition of the present invention comprises platinum and/or a platinum containing compound. In one aspect, the platinum or platinum containing compound is an active catalytic metal when impregnated and/or doped in a well dispersed manner over the silicoaluminophosphate molecular sieve zeolite support. The platinum content of the catalyst composition can range from about 0.1 wt. % to about 1.5 wt. %, for example, about 0.1, 0.2, 0.3, 0.4, 0.5, 0.6, 0.7, 0.8, 0.9, 1.0, 1.1, 1.2, 1.3, 1.4 or 1.5 wt. % platinum; from about 0.1 wt. % to about 1 wt. %; from about 0.2 wt. % to about 0.8 wt. %, for example, about 0.2, 0.3, 0.4, 0.5, 0.6, 0.7, or 0.8 wt. %; or from about 0.4 wt. % to about 0.6 wt. %, for example, about 0.4, 0.5, or 0.6 wt. % of the catalyst composition. In a specific aspect, the platinum content of the catalyst composition is about 0.5 wt. % of the catalyst composition. In one aspect, a support material can be contacted with a water soluble platinum species, such as, for example, chloroplatinic acid and/or other halogenated platinum and nitrates compounds. In another aspect, an organic platinum compound can be contacted with the support material. The concentration of any solution and/or suspension of a platinum compound can vary depending upon the specific target composition. In one aspect, a 0.03 M aqueous solution of H₂PtCl₆ can be contacted with a support material. After contacting the support material with a platinum containing compound, the resulting material can be dried, for example, at about 80° C. for about 3 hours, and then calcined, for example, at about 500° C. for about 4 hours. It should be noted that the specific temperature and time of any drying and/or calcining step can vary, and that one of skill in the art could readily determine an appropriate temperature and time. Platinum containing compounds are commercially available and one of skill in the art could readily select an appropriate platinum containing compound for use with the present invention.

The catalyst composition of the present invention also comprises a base promoter and one or more additional promoters. In one aspect, the base promoter comprises a tin component and a potassium component. In another aspect, the tin component comprises tin, a tin oxide, other tin containing compounds, or a combination thereof. In yet another aspect, the tin component comprises a tin oxide. The catalyst composition can comprise from about 0.4 wt. % to about 2 wt. %, for example, about 0.4, 0.5, 0.6, 0.7, 0.8, 0.9, 1, 1.1, 1.2, 1.3, 1.4, 1.5, 1.6, 1.7, 1.8, 1.9 or 2.0 wt. % tin; from about 0.5 wt. % to about 1.5 wt. %, for example, about 0.5, 0.6, 0.7, 0.8, 0.9, 1, 1.1, 1.2, 1.3, 1.4, or 1.5 wt. %; or from about 0.7 wt. % to about 1.2 wt. %, for example, 0.7, 0.8, 0.9, 1, 1.1 or 1.2 wt. % tin. In another aspect, the catalyst composition comprises about 0.9-1.0 wt. % tin. In one aspect, a support material can be contacted with a tin containing compound. In another aspect, a support material can be contacted with a water soluble tin containing compound. In other aspects, the tin containing compound can comprise a salt, such as, for example, a stannic chloride. The concentration of any solution and/or suspension of a tin compound can vary depending upon the specific target composition. In one aspect, a 0.15 M aqueous solution of SnCl₄ can be contacted with a support material. In another aspect, other tin containing compounds can be used and one of skill in the art could readily select an appropriate tin containing compound. Tin containing compounds, such as, for example, stannic chloride, are commercially available. As described above with respect to platinum, the support material can be dried and/or calcined after contacting with the tin containing compound.

In one aspect, the potassium component comprises potassium, a potassium oxide, other potassium containing compounds, or a combination thereof. In yet another aspect, the potassium component comprises a potassium oxide. The catalyst composition can comprise from about 0.2 wt. % to about 1.0 wt. %, for example, about 0.2, 0.3, 0.4, 0.5, 0.6, 0.7, 0.8, 0.9 or 1.0 wt. % potassium; from about 0.2 wt. % to about 0.8 wt. %, for example, about 0.2, 0.3, 0.4, 0.5, 0.6, 0.7, or 0.8 wt. %; or from about 0.4 wt. % to about 0.8 wt. %, for example, 0.4, 0.5, 0.6, 0.7, or 0.8 wt. % potassium. In another aspect, the catalyst composition comprises about 0.5-0.6 wt. % potassium. In one aspect, a support material can be contacted with a potassium containing compound. In another aspect, a support material can be contacted with a water soluble potassium containing compound. In other aspects, the potassium containing compound can comprise a salt, such as, for example, a potassium chloride. The concentration of any solution and/or suspension of a potassium compound can vary depending upon the specific target composition. In one aspect, a 0.03 M aqueous solution of KCl can be contacted with a support material. In another aspect, other potassium containing compounds can be used and one of skill in the art could readily select an appropriate potassium containing compound. Potassium containing compounds, such as, for example, potassium chloride, are commercially available. As described above with respect to platinum, the support material can be dried and/or calcined after contacting with the potassium containing compound.

The catalyst composition further comprises one or more additional promoters comprising a lanthanum component, a zinc component, a calcium component, a magnesium component, or a combination thereof. In another aspect, the catalyst composition comprises lanthanum and/or an oxide thereof, zinc and/or an oxide thereof, calcium and/or an oxide thereof, magnesium and/or an oxide thereof, or a combination thereof. In other aspects, other lanthanum, zinc, calcium, and/or magnesium containing compounds that can be impregnated after and/or before calcining a respective precursor and a support material can be present. Any such additional promoters, if present, can be added and/or impregnated before and/or after any or all of the principal promoters have been impregnated.

In one aspect, a lanthanum component, if present, comprises a lanthanum oxide. In one aspect, the catalyst composition does not comprise a lanthanum component. In another aspect, the catalyst composition comprises from about 0.2 wt. % to about 1 wt. %, for example, about 0.2, 0.3, 0.4, 0.5, 0.6, 0.7, 0.8, 0.9, or 1 wt. % lanthanum. In another aspect, the catalyst composition comprises about 0.6 wt. % lanthanum. In one aspect, a support material can be contacted with a lanthanum containing compound. In another aspect, a support material can be contacted with a water soluble lanthanum containing compound. The concentration of any solution and/or suspension of a lanthanum compound can vary depending upon the specific target composition. In one aspect, a 0.03 M aqueous solution of La(NO₃)₃.6H₂O can be contacted with a support material to provide the desired metallic content of lanthanum in the catalyst. In another aspect, other lanthanum containing compounds can be used and one of skill in the art could readily select an appropriate lanthanum containing compound. In one aspect, one or more water soluble lanthanum compounds can be utilized. Lanthanum containing compounds are commercially available. As described above with respect to platinum, the support material can be dried and/or calcined after contacting with the lanthanum containing compound.

In another aspect, a zinc component, if present, comprises a zinc oxide. In one aspect, the catalyst composition does not comprise a zinc component. In another aspect, the catalyst composition comprises from about 0.1 wt. % to about 1.0 wt. %, for example, about 0.1, 0.2, 0.3, 0.4, 0.5, 0.6, 0.7, 0.8, 0.9 or 1.0 wt. % zinc; or from about 0.1 wt. % to about 0.8 wt. %, for example, about 0.1, 0.2, 0.3, 0.4, 0.5, 0.6, 0.7, or 0.8 wt. % zinc. In another aspect, the catalyst composition comprises about 0.5-0.6 wt. % zinc. In one aspect, a support material can be contacted with a zinc containing compound. In another aspect, a support material can be contacted with a water soluble zinc containing compound, such as, for example, Zn(NO₃)₂. The concentration of any solution and/or suspension of a zinc compound can vary depending upon the specific target composition. In another aspect, other zinc containing compounds can be used and one of skill in the art could readily select an appropriate zinc containing compound. Zinc containing compounds are commercially available. As described above with respect to platinum, the support material can be dried and/or calcined after contacting with the zinc containing compound.

In another aspect, a calcium component, if present, comprises a calcium oxide. In one aspect, the catalyst composition does not comprise a calcium component. In another aspect, the catalyst composition comprises from about 0.1 wt. % to about 1 wt. %, for example, about 0.1, 0.2, 0.3, 0.4, 0.5, 0.6, 0.7, 0.8, 0.9, or 1 wt. % calcium. In another aspect, the catalyst composition comprises about 0.6 wt. % calcium. In one aspect, a support material can be contacted with a calcium containing compound. In another aspect, a support material can be contacted with a water soluble calcium containing compound, such as, for example, Ca₂(NO₃)₂. The concentration of any solution and/or suspension of a calcium compound can vary depending upon the specific target composition. In another aspect, other calcium containing compounds can be used and one of skill in the art could readily select an appropriate calcium containing compound. Calcium containing compounds are commercially available. As described above with respect to platinum, the support material can be dried and/or calcined after contacting with the calcium containing compound.

In another aspect, a magnesium component, if present, comprises a magnesium oxide. In one aspect, the catalyst composition does not comprise a magnesium component. In another aspect, the catalyst composition comprises from about 0.1 wt. % to about 1.2 wt. %, for example, about 0.1, 0.2, 0.3, 0.4, 0.5, 0.6, 0.7, 0.8, 0.9, 1, 1.1, or 1.2 wt. % magnesium. In another aspect, the catalyst composition comprises about 0.6 wt. % magnesium. In one aspect, a support material can be contacted with a magnesium containing compound. In another aspect, a support material can be contacted with a water soluble magnesium containing compound, such as, for example, Mg(NO₃)₂. The concentration of any solution and/or suspension of a magnesium compound can vary depending upon the specific target composition. In another aspect, other magnesium containing compounds can be used and one of skill in the art could readily select an appropriate magnesium containing compound. Magnesium containing compounds are commercially available. As described above with respect to platinum, the support material can be dried and/or calcined after contacting with the magnesium containing compound.

The catalyst composition can comprise a support material, platinum, a tin component, a potassium component, and one or more of a lanthanum component, a zinc component, a calcium component, and/or a magnesium component. In another aspect, the catalyst composition comprises two or more of a lanthanum component, a zinc component, a calcium component, and/or a magnesium component. In yet another aspect, the catalyst composition comprises three or more of a lanthanum component, a zinc component, a calcium component, and/or a magnesium component. In still another aspect, the catalyst composition comprises a lanthanum component, a zinc component, a calcium component, and a magnesium component. In one aspect, the platinum (active metal), tin component, potassium component, and the additional promoter can comprise a total metallic content from about 1.0 wt. % to about 5 wt. % of the catalyst composition.

The platinum (active metal), base promoter, and additional promoter components can be contacted individually in any order. In another aspect, any two or more components can be combined prior to or simultaneously with contacting, for example, a support material. In another aspect, the tin and potassium components can be contacted with the support material prior to a lanthanum component, a zinc component, a calcium component, or a magnesium component. In another aspect, the base and additional promoter components can be contacted in any order. In yet another aspect, the platinum containing compound can be contacted after the tin component, the potassium component, and any additional promoter components have been contacted with the support material. In still other aspects, the platinum containing compound can be contacted in any order deemed suitable for preparing a dehydrogenation catalyst composition.

In one aspect, any of the base metal and/or additional promoter components or a portion thereof are disposed on the surface of a support material. In another aspect, any of the base and/or additional promoter components or a portion thereof are impregnated in the support material. In still other aspects, all or substantially all of the base and/or additional promoter components are impregnated in the support material.

In one aspect, the ratio of promoter components, for example, tin component, potassium component, and an additional promoter components that are present (e.g., lanthanum, zinc, calcium, magnesium) to platinum is at least about 2:1.

After contacting a support material with any one or more promoters or platinum components, the resulting material can, in one aspect, be dried at a temperature of from about 70° C. to about 120° C. for a period of from about 1 to about 4 hours. In one aspect, such a drying step can be performed after each contacting step. In another aspect, multiple contacting steps can be performed prior to a drying step.

After contacting a support material with any one or more promoters or platinum components and optionally drying, the resulting material can, in one aspect, be calcined at a temperature of, for example, from about 400° C. to about 750° C. for a period of from about 1 to about 12 hours. In one aspect, such a calcining step can be performed after each contacting and/or drying step. In another aspect, multiple contacting steps and/or drying steps can be performed prior to a calcining step.

In yet another aspect, the catalyst composition can be dechlorinated with, for example, mild steam for a period of from about 2 minutes to about 60 minutes. In another aspect, the catalyst composition can be dechlorinated with mild steam for about 25 minutes. If such a dechlorination step is performed, the resulting catalyst can optionally be dried after dechlorination.

In another aspect, the catalyst composition can comprise a binder, such as, for example, an alumina binder.

In yet another aspect, the catalyst composition can be subjected to a reducing step to reduce any one or more of the platinum and/or promoter components impregnated thereon. A reducing step can be performed, for example, by contacting the catalyst composition with a flowing stream of hydrogen for a period of from about 2 minutes to about 30 minutes.

The catalyst composition of the present invention can be utilized as a stationary (fixed bed) dehydrogenation catalyst or moving bed or a fluid bed dehydrogenation catalyst. In another aspect, the catalyst composition can be utilized in a reactor comprising a membrane setup. In another aspect, the catalyst composition of the present invention can be utilized as a stationary catalyst in a membrane reactor. The catalyst composition is suitable for the conversion of alkanes, such as, for example, propane and butane, to olefins, for example, propylene and butylene.

The catalyst composition of the present invention can be used in any suitable reactor, such as, for example, a stationary (fixed bed) reactor or moving bed or a fluid bed reactor. In one aspect, the catalyst composition is used in a stationary reactor for the dehydrogenation of propane and/or butane. In another aspect, the catalyst composition is used in a fluid bed reactor for the dehydrogenation of propane and/or butane.

The catalyst composition can be contacted with an alkane or a mixture of alkanes to at least partially dehydrogenate the alkane or mixture of alkanes and provide a desirable olefin. In one aspect, the catalyst composition is contacted with an alkane stream comprising propane, butane, and/or a mixed feed, and/or an alkane feed with light and heavy impurities. In such an aspect, the catalyst composition can convert at least a portion of the propane and butane to propylene and butylene, respectively. In another aspect, the catalyst composition is used in a direct dehydrogenation reaction. In still another aspect, the catalyst composition can be used in an oxydehydrogenation reaction and/or for a hydrogenation reaction. In one aspect, the catalyst composition is used in a non-oxydehydrogenation reaction. Thus, in one aspect, the reaction environment is free of or substantially free of oxygen, such that direct dehydrogenation occurs. For example, the process can be direct dehydrogenation without oxygen.

In another aspect, the catalyst composition is used with process conditions comprising a WHSV of from about 0.1/h to about 50/h, for example, about 0.1, 0.2, 0.3, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5 or 50/hr; from about 0.1/h to about 20/h, for example, about 0.1, 0.2, 0.3, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20/h; or from about 1/h to about 10/h, for example, about 0.1, 0.2, 0.3, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.5, 5, 5.5, 6, 6.5, 7, 7.5, 8, 8.5, 9, 9.5, or 10/h. In another aspect, the catalyst composition is used with process conditions comprising a WHSV of less than about 20/h. In another aspect, the catalyst composition is used with process conditions for oxydehydrogenation and hydrogenation reactions, the WHSV varies, as in hydrogenation WSHV is about 100-10000/h.

In yet another aspect, the catalyst composition is used with process conditions comprising a temperature of from about 500° C. to about 650° C., for example, about 500, 510, 520, 530, 540, 550, 560, 570, 580, 590, 600, 610, 620, 630, 640, or 650° C.; or from about 550° C. to about 650° C., for example, about 550, 560, 570, 580, 590, 600, 610, 620, 630, 640, or 650° C. In another aspect, the catalyst composition is used with process conditions comprising a temperature of about 590° C. In another aspect, the catalyst composition is used at low pressure, for example, less than about 2 atm.

In various aspects, the catalyst of the present invention is robust and can exhibit high and stable activity, propylene selectivity, as well as high hydrothermal stability.

In conventional endothermic alkane dehydrogenation units, a continuous supply of heat during the reaction is a key factor in design choices of the reactor and their cyclic process. In one aspect, the catalyst composition of the present invention provides enhanced heat stability of the catalyst bed and reduced pressure drop issues in the reactor, as compared to a conventional dehydrogenation catalyst.

In one aspect, the catalyst composition can provide a selectivity for propylene production of at least about 88%, for example, 88, 89, 90, 91, 92, 93, 94, 95%, or greater; at least about 90%, or at least about 92%, for example, 92, 93, 94, 95% or greater.

The catalyst composition can be used in any process conditions suitable for the conversion of alkanes to olefins. In one aspect, an alkane or mixture of alkanes is contacted with the catalyst composition described herein so as to convert at least a portion of the alkane or mixture of alkanes to olefins. In another aspect, the catalyst composition is contacted with an alkane stream comprising propane and/or butane, so as to convert at least a portion of the alkane stream to propylene and/or butylene.

In one aspect, the catalyst composition exhibits high catalytic activity as compared to a convention dehydrogenation catalyst. In another aspect, the catalyst composition exhibits improved hydrothermal stability as compared to a conventional dehydrogenation catalyst. In still another aspect, the catalyst composition can provide a high degree of selectivity as compared to a conventional dehydrogenation catalyst.

Set forth below are some embodiments of the catalyst composition and method disclosed herein.

### Embodiment 1

A light alkane dehydrogenation catalyst composition comprising: about 0.1 wt. % to about 1.5 wt. % platinum, based on the total amount of the catalyst composition; b) a base promoter comprising a tin component and a potassium component; c) an additional promoter comprising a lanthanum component, a zinc component, a calcium component, a magnesium component, or a combination thereof; and d) a silicoaluminophosphate zeolite molecular sieve support.

### Embodiment 2

The dehydrogenation catalyst composition of Embodiment 1, wherein the base promoter comprises tin and/or an oxide thereof, and potassium and/or an oxide thereof.

### Embodiment 3

The dehydrogenation catalyst composition of any of Embodiments 1-2, wherein the additional promoter comprises lanthanum and/or an oxide thereof, zinc and/or an oxide thereof, calcium and/or an oxide thereof, magnesium and/or an oxide thereof, or a combination thereof.

### Embodiment 4

The dehydrogenation catalyst composition of any of claims 1-3, wherein the dehydrogenation catalyst composition provides an olefin selectivity of at least about 90%.

### Embodiment 5

The dehydrogenation catalyst composition of any of claims 1-4, wherein the selectivity to propylene production from alkanes is at least about 88%.

### Embodiment 6

The dehydrogenation catalyst composition of any of Embodiments 1-5, comprising from about 0.2 wt. % to about 0.8 wt. % platinum.

### Embodiment 7

The dehydrogenation catalyst composition of any of Embodiments 1-6, comprising from about 0.4 wt. % to about 2 wt. % tin.

### Embodiment 8

The dehydrogenation catalyst composition of any of Embodiments 1-7, comprising from about 0.7 wt. % to about 1.1 wt. % tin.

### Embodiment 9

The dehydrogenation catalyst composition of any of Embodiments 1-8, comprising from about 0.2 wt. % to about 1 wt. % potassium.

### Embodiment 10

The dehydrogenation catalyst composition of any of Embodiments 1-9, comprising from about 0.4 wt. % to about 0.8 wt. % potassium.

### Embodiment 11

The dehydrogenation catalyst composition of any of Embodiments 1-10, comprising from about 0.2 wt. % to about 1 wt. % lanthanum.

### Embodiment 12

The dehydrogenation catalyst composition of any of Embodiments 1-11, comprising from about 0.1 wt. % to about 1 wt. % zinc.

### Embodiment 13

The dehydrogenation catalyst composition of any of Embodiments 1-12, comprising from about 0.1 wt. % to about 1 wt. % calcium.

### Embodiment 14

The dehydrogenation catalyst composition of any of Embodiments 1-13, comprising from about 0.1 wt. % to about 1.2 wt. % magnesium.

### Embodiment 15

The dehydrogenation catalyst composition of any of Embodiments 1-14, wherein the total metallic content of the catalyst composition, including platinum, tin, potassium, and the additional promoter, is from about 1 wt. % to about 5 wt. % of the catalyst composition.

### Embodiment 16

The dehydrogenation catalyst composition of any of Embodiments 1-15, wherein c) comprises at least two additional promoters comprising lanthanum, zinc, calcium, magnesium, or a combination thereof.

### Embodiment 17

The dehydrogenation catalyst composition of any of Embodiments 1-16, wherein c) comprises at least three additional promoters comprising lanthanum, zinc, calcium, magnesium, or a combination thereof.

### Embodiment 18

The dehydrogenation catalyst composition of any of Embodiments 1-17, comprising lanthanum, zinc, calcium, and magnesium promoters.

### Embodiment 19

The dehydrogenation catalyst composition of any of Embodiments 1-18, wherein a ratio of tin, potassium, and the additional promoter to platinum is at least about 2:1.

### Embodiment 20

The dehydrogenation catalyst composition of any of Embodiments 1-19, wherein the support comprises a silicoaluminophosphate zeolite molecular sieve.

### Embodiment 21

The dehydrogenation catalyst composition of any of Embodiments 1-20, wherein the support comprises one or more of AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, ALPO-17, ALPO-18, ALPO-34, SAPO-5, SAPO-17, SAPO-18, SAPO-34, SAPO-35, SBA-15, MCM-42, ZK-4, ZSM-2, ZSM-5, ZK-14, SAPO-42, ZK-21, ZK-22, ZK-5, ZK-20, zeolite A, hydroxysodalite, erionite, chabazite, zeolite T, gmelinite, clinoptilolite, alumina bound and/or surface coated zeolites, substituted groups thereof, or a combination thereof.

### Embodiment 22

The dehydrogenation catalyst composition of any of Embodiments 1-21, wherein the silicoaluminophosphate zeolite molecular sieve support comprises SAPO-34.

### Embodiment 23

The dehydrogenation catalyst composition of any of Embodiments 1-22, wherein the silicoaluminophosphate zeolite molecular sieve support is modified with an aluminum containing compound.

### Embodiment 24

The dehydrogenation catalyst composition of any of Embodiments 1-23, wherein the support comprises a silicoaluminophosphate zeolite molecular sieve support, e.g., a small pore size silicoaluminophosphate zeolite molecular sieve support.

### Embodiment 25

The dehydrogenation catalyst composition of any of Embodiments 1-24, wherein the silicoaluminophosphate zeolite molecular sieve support is modified with alumina.

### Embodiment 26

The dehydrogenation catalyst composition of any of Embodiments 1-25, wherein the silicoaluminophosphate zeolite molecular sieve support is modified with an aluminum source compound.

### Embodiment 27

The dehydrogenation catalyst composition of any of Embodiments 1-26, wherein the silicoaluminophosphate zeolite molecular sieve support is modified with kaolin.

### Embodiment 28

The dehydrogenation catalyst composition of any of Embodiments 1-27, wherein at least a portion of the tin, potassium, and the additional promoter is impregnated into a surface of the support.

### Embodiment 29

A method for preparing a catalyst composition, the method comprising contacting a support material with a tin component, a potassium component; one or more of a lanthanum component, a zinc component, a calcium component, a magnesium component, or a combination thereof.

### Embodiment 30

The method of Embodiment 29, wherein a ratio of the tin component, potassium component, and one or more of a lanthanum component, zinc component, calcium component, and magnesium component to platinum is at least about 2:1

### Embodiment 31

The method of any of Embodiments 29-30, wherein after contacting the support material with the tin component, the potassium component, and/or one or more of the lanthanum component, the zinc component, the calcium component, and the magnesium component, the resulting material is dried at a temperature of from about 70° C. to about 120° C. for a period of from about 1 to about 4 hours.

### Embodiment 32

The method of any of Embodiments 29-31, wherein after contacting the support material with the tin component, the potassium component, and/or one or more of the lanthanum component, the zinc component, the calcium component, and the magnesium component, the resulting material, the resulting material is calcined at a temperature of from about 400° C. to about 750° C. for a period of from about 1 to about 12 hours.

### Embodiment 33

The method of any of Embodiments 29-32, further comprising dechlorinating the catalyst composition with steam, e.g., mild steam.

### Embodiment 34

The method of any of Embodiments 29-33, further comprising subjecting the catalyst composition to a reducing step.

### Embodiment 35

The method of Embodiment 34, wherein the reducing step comprises exposing the catalyst composition to flowing hydrogen.

### Embodiment 36

A method for converting an alkane to an olefin, the method comprising contacting the alkane with the catalyst composition of any of Embodiments 1-28.

### Embodiment 37

The method of Embodiment 36, wherein the alkane comprises propane and/or butane.

### Embodiment 38

The method of any of Embodiments 36-37, wherein a reaction environment is free of or substantially free of oxygen.

### Embodiment 39

The method of any of Embodiments 36-38, wherein the reaction is a direct dehydrogenation reaction.

### Embodiment 40

The method of any of Embodiments 36-39, wherein the catalyst composition provides a propylene selectivity of at least about 88%.

### Embodiment 41

The method of any of Embodiments 36-40, wherein the catalyst composition provides an olefin selectivity of at least about 94%.

### Embodiment 42

The method of any of Embodiments 36-41, wherein the catalyst composition is disposed in a stationary fixed-bed reactor.

### Embodiment 43

The method of any of Embodiments 36-41, wherein the catalyst composition is disposed in a fluidized bed reactor.

### Embodiment 44

The method of any of Embodiments 36-41, wherein the catalyst composition is disposed in a moving bed reactor.

### Embodiment 45

The method of any of Embodiments 36-41, wherein the catalyst composition is disposed in a membrane reactor.

### Embodiment 46

The method of any of Embodiments 36-41, wherein the catalyst composition is disposed in one or more membranes integrated in a fixed bed, moving bed, and/or fluidized bed reactor.

### Embodiment 47

The method of any of Embodiments 36-46, wherein the method comprises dehydrogenation of light alkane.

### Embodiment 48

The method of any of Embodiments 36-47, wherein process conditions comprise a WHSV of from 0.1/h to about 50/h.

### Embodiment 49

The method of any of Embodiments 36-48, wherein process conditions comprise a WHSV of less than about 20/h.

### Embodiment 50

The method of any of Embodiments 36-49, wherein process conditions for propane dehydrogenation comprise a WHSV of from about 1/h to about 10/h.

### Embodiment 51

The method of any of Embodiments 36-50, wherein process conditions for propane dehydrogenation comprise a temperature of from about 500° C. to about 650° C.

### Embodiment 52

The catalyst composition of any of Embodiments 1-28, having improved hydrothermal stability as compared to a conventional dehydrogenation catalyst.

### Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Preparation of Dehydrogenation Catalysts

In a first example, a dehydrogenation catalyst composition was prepared by sequential impregnation of metals onto a powdered SAPO-34 zeolite support. The SAPO-34 support was first prepared by mixing Al₂O₃/P₂O₅/SiO₂/TEA/H₂O in a molar ratio of 1:1:0.5:2:100. The SAPO-34 support was then sequentially impregnated with aqueous solutions of 0.03 M KCl, 0.15 M SnCl₄ and 0.03 M H₂PtCl₆ to provide a final concentration of 0.5 wt. % Pt, 0.9 wt. % Sn, and 0.6 wt % K in the final catalyst composition. After each impregnation step, the prepared samples were dried at 80° C. for 3 hr and then calcined at 500° C. for 4 hr. This sample is referenced as Catalyst A in Table 1, below. Additional catalyst compositions (B—I) were then prepared by sequentially impregnating additional promoters. The details of each catalyst composition are listed in Table 1, below. Catalyst B (Pt—Sn—K—La/SAPO-34) was prepared by sequential impregnation as with Catalyst A, with additional 0.6 wt % La in the final catalyst. Catalyst C (Pt—Sn—K—Ca/SAPO-34) was prepared by sequential impregnation as with Catalyst A, with additional 0.6 wt % Ca in the final catalyst. Catalyst D (Pt—Sn—K—Mg/SAPO-34) was prepared by sequential impregnation as with Catalyst A, with additional 0.5 wt % Mg in final catalyst. Catalyst E (Pt—Sn—K—Zn/SAPO-34) was prepared by sequential impregnation as with Catalyst A, with additional 0.6 wt % Zn in the final catalyst. Catalyst F (Pt—Sn—K—Ca—La/SAPO-34) was prepared by sequential impregnation as with Catalyst C, with additional 0.6 wt % La in the final catalyst. Catalyst G (Pt—Sn—K—Ca—Zn/SAPO-34) was prepared by sequential impregnation as with Catalyst C, with additional 0.6 wt % Zn in the final catalyst. Catalyst H (Pt—Sn—K—Ca—La—Mg/SAPO-34) was prepared by sequential impregnation as with Catalyst F, with additional 0.5 wt % Mg in the final catalyst. Catalyst I (Pt—Sn—K—Ca—La—Zn/SAPO-34) was prepared, by sequential impregnation as with Catalyst F, with additional 0.6 wt % Zn in the final catalyst. The metallic contents were confirmed in the final catalysts by using XRF.

TABLE 1
Multi-promoter intensified Pt-based SAPO-34 supported catalyst.

| | Support | Active Metals and Promoters (wt. %) | | | | | | |
|---|---|---|---|---|---|---|---|---|
| Catalyst | SAPO-34 | Pt | Sn | K | La | Ca | Mg | Zn |
| A | | 0.5 | 0.9 | 0.6 | — | — | — | — |
| B | | | | | 0.6 | — | — | — |
| C | | | | | — | 0.6 | — | — |
| D | | | | | — | — | 0.5 | — |
| E | | | | | — | — | — | 0.6 |
| F | | | | | 0.6 | 0.6 | — | — |
| G | | | | | — | 0.6 | — | 0.6 |
| H | | | | | 0.6 | 0.6 | 0.5 | — |
| I | | | | | 0.6 | 0.6 | — | 0.6 |

2. Preparation of Dehydrogenation Catalysts on Modified Supports

In a second example, dehydrogenation catalyst compositions were prepared by sequential impregnation of metals onto a modified SAPO-34 zeolite support. The modified-SAPO-34 support was prepared using a non-mesopore template, i.e. kaolin (a combined source of aluminum and silicon), phosphorus, template, and de-ionized water mixed together and stirred to obtain a uniform crystallization solution. The kaolin:P₂O₅:H₂O were mixed in a molar ratio of ˜1.5:1:500, and then the mixture was subsequently stirred, aged, autoclaved, filtered, washed, dried, and calcined at 600° C., wherein a silicoalumino phosphate catalyst with a slit shape was obtained.

Catalyst J (Pt—Sn—K/Modefied-SAPO-34) was prepared by sequential impregnation of metals on the modified-SAPO-34 zeolite. The modified-SAPO-34 was impregnated with aqueous solutions of 0.03M KCl, 0.15 SnCl₄ and 0.03 M H₂PtCl₆ to provide final concentrations of 0.5 wt. % Pt, 0.9 wt. % Sn, and 0.6 wt % of K in the final catalyst, respectively. After each impregnation step, the prepared samples were dried at 80° C. for 3 hr and then calcined at 500° C. for 4 hr. The details of each modified support catalyst composition (J-Q) are listed in Table 2, below. Catalyst K (Pt—Sn—K—La/modified-SAPO-34) was prepared by sequential impregnation as with Catalyst J, with additional 0.6 wt % La in the final catalyst. Catalyst L (Pt—Sn—K—Ca/modified-SAPO-34) was prepared by sequential impregnation as with Catalyst J, with additional 0.6 wt % Ca in the final catalyst. Catalyst M (Pt—Sn—K—Zn/modified-SAPO-34) was prepared by sequential impregnation as with Catalyst J, with additional 0.6 wt % Zn in final catalyst. Catalyst N (Pt—Sn—K—Ca—La/modified-SAPO-34) was prepared by sequential impregnation as with Catalyst L, with additional 0.6 wt % La in the final catalyst. Catalyst 0 (Pt—Sn—K—Ca—Zn/modified-SAPO-34) was prepared by sequential impregnation as with Catalyst L, with additional 0.6 wt % Zn in the final catalyst. Catalyst P (Pt—Sn—K—Ca—La—Zn/modified-SAPO-34) was prepared by sequential impregnation as with Catalyst 0, with additional 0.6 wt % La in final catalyst. Catalyst Q (Pt—Sn—K—La—Zn/modified-SAPO-34) was prepared by sequential impregnation as with Catalyst K, with additional 0.6 wt % Zn in final catalyst.

The X-ray diffraction (XRD) pattern of a Pt—Sn—K/Modified-SAPO-34 (Catalyst J) was obtained using a powder X-ray diffractometer, in order to ensure texture of support as illustrated in FIG. 1. The surface morphology of the modified-SAPO-34 support (Slit-SAPO-34) was characterized using scanning electron microscope (SEM) and is illustrated in the inset (upper portion) of FIG. 1. This XRD pattern is similar to SAPO-34, indicating that only the surface topology is modified.

The Brönsted acid sites of Pt—Sn—K/SAPO-34 (Catalyst A) and Pt—Sn—K/Modified-SAPO-34 (Catalyst J) were determined by NH₃-TPD, using a 0.2 g sample for passing ammonia and desorb at 100° C., and is between 2.1-2.35. The ability of the catalysts to adsorb hydrogen was also determined by hydrogen pulse chemisorption using a conventional setup to measure active Pt sites available for dehydrogenation and total hydrogen uptake at various temperatures. The results of such analysis was about 50-60 ml H₂/g Pt.

TABLE 2
Pd-based modified SAPO-34 catalyst with different promoters and
combinations.

| | Support | Active Metals and Promoters (wt. %) | | | | | |
|---|---|---|---|---|---|---|---|
| Catalyst | Modefied-SAPO-34 | Pt | Sn | K | La | Ca | Zn |
| J | | 0.5 | 0.9 | 0.6 | — | — | — |
| K | | | | | 0.6 | — | — |
| L | | | | | — | 0.6 | — |
| M | | | | | — | — | 0.6 |
| N | | | | | 0.6 | 0.6 | — |
| O | | | | | — | 0.6 | 0.6 |
| P | | | | | 0.6 | 0.6 | 0.6 |
| Q | | | | | 0.6 | — | 0.6 |

3. Performance for Direct Propane Dehydrogenation to Propylene

In a third example, the performance of inventive catalyst compositions A-Q, as described in Examples 1 and 2, were evaluated in an Iso-thermal Fixed Bed Reactor at WHSV 5.5/hr and 590° C. The conversion efficiency and selectivity for each catalyst are detailed in Table 3, below, for 1 hr Time on Stream (TOS) and 5 hr TOS.

TABLE 3
Catalysts Performance for Propane dehydrogenation to
Propylene in Iso-thermal Fixed Bed Reactor at WHSV
5.5/hr and 590° C.

| | TOS = 1 hr | | TOS = 5 hr | |
|---|---|---|---|---|
| Catalyst | Conversion | Selectivity | Conversion | Selectivity |
| A | 33.1 | 93.2 | 24.4 | 94.6 |
| B | 34.2 | 94.1 | 25.1 | 94.5 |
| C | 35.0 | 94.3 | 25.9 | 95.1 |
| D | 31.2 | 93.8 | 22.3 | 95.7 |
| E | 31.4 | 94.2 | 26.1 | 95.2 |
| F | 36.4 | 94.3 | 26.2 | 96.1 |
| G | 32.8 | 93.9 | 22.8 | 96.1 |
| H | 35.1 | 94.6 | 25.4 | 95.8 |
| I | 36.7 | 94.5 | 26.6 | 96.5 |
| J | 36.3 | 91.2 | 25.9 | 92.1 |
| K | 36.7 | 91.1 | 25.7 | 92.2 |
| L | 37.1 | 91.8 | 26.2 | 93.2 |
| M | 33.3 | 91.6 | 27.2 | 93.1 |
| N | 36.8 | 91.5 | 27.1 | 93.4 |
| O | 35.8 | 90.9 | 27.4 | 93.5 |
| P | 35.7 | 91.3 | 26.9 | 93.2 |
| Q | 37.3 | 91.8 | 27.3 | 94.1 |

4. Comparison of Conventional and Inventive Catalyst Compositions

In a fourth example, the performance of conventional and inventive catalyst compositions was evaluated for propane dehydrogenation to propylene in an Iso-thermal Dixed Bed Reactor at WHSV 5.5/hr. Conversion and Selectivity values for each catalyst are detailed in Table 4, below.

TABLE 4
Comparison of Prior art Catalysts with Current Invention for Propane
dehydrogenation to Propylene in Iso-thermal Fixed Bed Reactor at
WHSV 5.5/hr.

| | Conventional Catalysts | | | | | |
|---|---|---|---|---|---|---|
| | Pt—Sn/ | | | | | |
| | SAPO- | Pt—Sn—La/ | Pt—Sn—Ca/ | Inventive Catalysts | | |
| Catalysts | 34 | SAPO-34 | SAPO-34 | A | F | I |

| | | | | |
|---|---|---|---|---|
| Example | — | 1 | 6 | 9 |

At TOS = 1 hr and Temperature 590° C.

| | | | | | | |
|---|---|---|---|---|---|---|
| Conversion | 20.1 | 31.7 | 34.3 | 34.1 | 36.5 | 36.8 |
| Selectivity | 89.1 | 95 | 94.1 | 94.2 | 94.4 | 94.8 |

At TOS = 2 hr and Temperature 590° C.

| | | | | | | |
|---|---|---|---|---|---|---|
| Conversion | 18.1 | 27.9 | 31.8 | 33.3 | 33.9 | 34.1 |
| Selectivity | 91.6 | 94.3 | 94.6 | 94.9 | 95.3 | 95.7 |

At TOS = 5 hr and Temperature 590° C.

| | | | | | | |
|---|---|---|---|---|---|---|
| Conversion | 15.2 | 22.3 | 21.8 | 25.4 | 26.2 | 26.8 |
| Selectivity | 92.8 | 95.5 | 95.1 | 95.6 | 96.2 | 96.7 |

Similarly, the performance of several inventive catalyst compositions was evaluated for the direct dehydrogenation of butane to butylene, as detailed in Table 5, below.

TABLE 5
Catalysts Performance for n-Butane dehydrogenation to Butylene in
Iso-thermal Fixed Bed Reactor at WHSV 5.5/hr and 590° C.

| | TOS = 1 hr | | TOS = 5 hr | |
|---|---|---|---|---|
| Catalyst | Conversion | Selectivity | Conversion | Selectivity |
| F | 31.1 | 82.4 | 25.3 | 83.1 |
| I | 32.2 | 82.7 | 26.1 | 83.7 |
| N | 35.1 | 82.3 | 26.3 | 83.9 |
| O | 35.8 | 81.9 | 26.4 | 84.1 |
| P | 36.7 | 82.1 | 28.2 | 84.5 |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A light alkane dehydrogenation catalyst composition comprising
a. about 0.1 wt. % to about 1.5 wt. % platinum, based on the total amount of the catalyst composition;
b. a base promoter comprising a tin component and a potassium component;
c. an additional promoter comprising a lanthanum component, a zinc component, a calcium component, a magnesium component, or a combination thereof; and
d. a silicoaluminophosphate zeolite molecular sieve support.

2. The dehydrogenation catalyst composition of claim 1, wherein the base promoter comprises tin and/or an oxide thereof, and potassium and/or an oxide thereof.

3. The dehydrogenation catalyst composition of claim 1, wherein the additional promoter comprises lanthanum and/or an oxide thereof, zinc and/or an oxide thereof, calcium and/or an oxide thereof, magnesium and/or an oxide thereof, or a combination thereof.

4. The dehydrogenation catalyst composition of claim 1, comprising from about 0.2 wt. % to about 0.8 wt. % platinum; and/or from about 0.4 wt. % to about 2 wt. % tin; and/or from about 0.2 wt. % to about 1 wt. % potassium; and/or from about 0.2 wt. % to about 1 wt. % lanthanum; and/or from about 0.1 wt. % to about 1 wt. % zinc; and/or from about 0.1 wt. % to about 1 wt. % calcium.

5. The dehydrogenation catalyst composition of claim 1, comprising from about 0.1 wt. % to about 1.2 wt. % magnesium.

6. The dehydrogenation catalyst composition of claim 1, wherein the total metallic content of the catalyst composition, including platinum, tin, potassium, and the additional promoter, is from about 1 wt. % to about 5 wt. % of the catalyst composition.

7. The dehydrogenation catalyst composition of claim 1, wherein c) comprises at least two additional promoters comprising lanthanum, zinc, calcium, magnesium, or a combination thereof.

8. The dehydrogenation catalyst composition of claim 1, wherein a ratio of tin, potassium, and the additional promoter to platinum is at least about 2:1.

9. (canceled)

10. (canceled)

11. The dehydrogenation catalyst composition of claim 1, wherein the silicoaluminophosphate zeolite molecular sieve support is modified with an aluminum containing compound and/or with kaolin.

12. (canceled)

13. The dehydrogenation catalyst composition of claim 1, wherein at least a portion of the tin, potassium, and the additional promoter is impregnated into a surface of the support.

14. A method for preparing a catalyst composition, the method comprising contacting a support material with a platinum component, a tin component, a potassium component; one or more of a lanthanum component, a zinc component, a calcium component, a magnesium component, or a combination thereof.

15. The method of claim 14, wherein a ratio of the tin component, potassium component, and one or more of a lanthanum component, zinc component, calcium component, and magnesium component to platinum is at least about 2:1

16. The method of claim 14, wherein after contacting the support material with the tin component, the potassium component, and/or one or more of the lanthanum component, the zinc component, the calcium component, and the magnesium component, the resulting material is dried at a temperature of from about 70° C. to about 120° C. for a period of from about 1 to about 4 hours.

17. The method of claim 14, wherein after contacting the support material with the tin component, the potassium component, and/or one or more of the lanthanum component, the zinc component, the calcium component, and the magnesium component, the resulting material, the resulting material is calcined at a temperature of from about 400° C. to about 750° C. for a period of from about 1 to about 12 hours.

18. The method of claim 14, further comprising dechlorinating the catalyst composition with steam; and/or subjecting the catalyst composition to a reducing step.

19. A method for converting an alkane to an olefin, the method comprising contacting the alkane with a dehydrogenation catalyst composition, the dehydrogenation catalyst composition comprising
a. about 0.1 wt. % to about 1.5 wt. % platinum, based on the total amount of the catalyst composition;
b. a base promoter comprising a tin component and a potassium component;
c. an additional promoter comprising a lanthanum component, a zinc component, a calcium component, a magnesium component, or a combination thereof; and
d. a silicoaluminophosphate zeolite molecular sieve support.

20. The method of claim 19, wherein the alkane comprises propane and/or butane; and wherein a reaction environment is free of or substantially free of oxygen.

21. The method of claim 19, wherein the reaction is a direct dehydrogenation reaction, and wherein the method comprises dehydrogenation of light alkane.

22. (canceled)

23. The method of claim 19, wherein process conditions comprise a WHSV of from 0.1/h to about 50/h.

24. The method of claim 19, wherein process conditions for propane dehydrogenation comprise a temperature of from about 500° C. to about 650° C.
